(19) United States
(12) Patent Application Publication
(10) Pub. No.: US 2011/0060242 A1
(43) Pub. Date: Mar. 10, 2011

(54) Systems and methods for intra-operative stimulation within a surgical field

(75) Inventors: MICHAEL R. HAUSMAN, NEW YORK, NY (US); GEOFFREY B. THROPE, SHAKER HEIGHTS, OH (US); ROBERT B. STROTHER, WILLOUGHBY HILLS, OH (US); JOSEPH J. MRVA, EUCLID, OH (US)

(73) Assignee: Checkpoint Surgical, LLC

(21) Appl. No.: 12/806,697

(22) Filed: Aug. 19, 2010

Related U.S. Application Data
(63) Continuation-in-part of application No. 11/651,165, filed on Jan. 9, 2007.
Application No. 11/651,165 is a continuation-in-part of application No. 11/099,848, filed on Apr. 6, 2005.
(60) Provisional application No. 60/657,277, filed on Mar. 1, 2005.
(60) Provisional application No. 61/338,312, filed on Feb. 16, 2010.

(51) Int. Cl. A61B 5/05

(52) U.S. Cl. 600/554

(57) ABSTRACT

Improved assemblies, systems, and methods provide safeguarding against tissue injury during surgical procedures and/or identify nerve damage occurring prior to surgery and/or verify range of motion or attributes of muscle contraction during reconstructive surgery. Such improved assemblies may be utilized in embodiments of methods according to the present invention allowing, preferably by a single hand, manipulation, carrying, parameter adjustment and/or informational feedback generation from within a surgical field. Single-handed operation and easily identifiable feedback generation is desirable especially where focused attention is required, such as during surgery observed through a microscope.

### RELATED APPLICATIONS

This application is a continuation-in-part of co-pending U.S. patent application Ser. No. 11/651,165, filed Jan. 9, 2007, and entitled “Systems and Methods for Intra-Operative Stimulation,” which is a continuation-in-part of U.S. patent application Ser. No. 11/099,848, filed Apr. 6, 2005, and entitled “Systems and Methods for Intra-Operative Stimulation,” which claims the benefit of U.S. Provisional Patent Application Ser. No. 60/657,277, filed Mar. 1, 2005, and entitled “Systems and Methods for Intra-Operative Stimulation,” each of which is incorporated herein by reference in its entirety.

This application also claims the benefit of U.S. Patent Application Ser. No. 61/338,312, filed Feb. 16, 2010, and entitled “Systems and Methods for Intra-Operative Stimulation,” which is incorporated herein by reference in its entirety.

### FIELD OF THE INVENTION

The invention relates generally to tissue identification and integrity testing, and more particularly to systems and methods for safeguarding against nerve and muscle injury during surgical procedures, location and stimulation of nerves and muscles, identification and assessment of nerve and muscle integrity following traumatic injuries, and verification of range of motion and attributes of muscle contraction during reconstructive surgery.

### BACKGROUND OF THE INVENTION

Even with today's sophisticated medical devices, surgical procedures are not risk-free. Each patient's anatomy differs, requiring the surgeon to be ever vigilant to these differences so that the intended result is accomplished. The positioning of nerves and other tissues within a human or animal's body is one example of how internal anatomy differs from patient to patient. While these differences may be slight, if the surgeon fails to properly identify one or several nerves, the nerves may be bruised, stretched, or even severed during an operation. The negative effects of nerve damage can range from lack of feeling on that part of the body to loss of muscle control.

Traumatic injuries often require surgical repair. Determining the extent of muscle and nerve injury is not always possible using visual inspection. Use of an intra-operative stimulator enables accurate evaluation of the neuromuscular system in that area. This evaluation provides valuable knowledge to guide repair/reconstructive surgery following traumatic injury, and when performing a wide range of surgeries.

### SUMMARY OF THE INVENTION

The invention provides devices, systems, and methods for intra-operative stimulation. The intra-operative stimulation enables accurate evaluation of the neuromuscular system to guide repair or reconstructive surgery.

An embodiment of a method according to the present invention is a method of applying electrical stimulation to animal tissue. The method includes the step of identifying a three-dimensional surgical field comprising a targeted tissue region of an animal. The targeted tissue region may include muscle and/or nerve tissue. A device is provided, which includes a housing extending along a housing longitudinal axis between a housing proximal end and a housing distal end and electrical stimulation generation circuitry at least substantially contained within the housing. The device is preferably at least substantially sterile. Also provided are one or more user operable controls coupled to the housing and in operational communication with the electrical stimulation generation circuitry. Extending from the housing proximal end is an operative element comprising an electrode operatively coupled to the electrical stimulation generation circuitry. Also substantially contained within the housing is a power supply that is electrically coupled to the electrical stimulation generation circuitry. The device may be entirely inserted or introduced into the surgical field, and an electrical stimulation, generated by the electrical stimulation generation circuitry and delivered through the electrode may be applied to at least a portion of the targeted tissue region. This applying step may be carried out while the entire device or device housing is placed entirely within the surgical field.

According to one aspect of the invention, the surgical field, which may be at least substantially sterile, may extend from the targeted tissue region for a maximum length of thirty centimeters. Additionally or alternatively, the surgical field may be about 20 to about 50 millimeters wide, about 18 to about 22 centimeters long, and about 20 to about 50 millimeters deep.

According to another aspect of the invention, a method may further include the step of carrying the device by a single human hand. Preferably, the single human hand may be used to manipulate the device to change an electrical stimulation parameter of a stimulation to be generated by the electrical stimulation generation circuitry. Even more preferably, the manipulation may be carried out while the entire housing is positioned within the surgical field.

According to still another aspect of the present invention, the provided device may further include an electronic visual indicator operatively coupled to the stimulation circuitry. In performing a method according to the present invention, a first visual indication provided by the visual indicator may be observed, where the first visual indication is indicative of electrical power supplied to the stimulation circuitry by the power supply. Further, a second visual indication provided by the visual indicator may be observed, where the second visual indication is indicative of electrical stimulation flowing at least partially through the targeted tissue region. Where two visual indications may be observed, they may be different. For example, the first visual indication may be an illumination of a first color and the second visual indication may be an illumination of a second color, the second color being different from the first color. Additionally, or alternatively, the illuminations may be of a different flash pattern, such as being illuminated continuously, or having a flashing pattern.

According to yet another aspect of the present invention, the visual indicator may be an illumination device that is radially visible from 360 degrees around the longitudinal axis of the handle, and it may be situated between one of the user operable controls and the electrode.

In another embodiment of a method according to the present invention, a method of applying electrical stimulation to a targeted animal tissue region includes the step of receiving, into a single human hand, a device. The device includes a housing extending along a housing longitudinal axis between a housing proximal end and a housing distal end, electrical stimulation generation circuitry at least substantially contained within the housing, and one or more user operable controls coupled to the housing and in operational communication with the electrical stimulation generation circuitry. Extending from the proximal end of the housing is an operative element which includes an electrode operatively coupled to the electrical stimulation generation circuitry. Also contained at least substantially within the housing is a power supply that is electrically coupled to the electrical stimulation generation circuitry. The method further includes the step of generating a first indication, which is indicative of electrical power being supplied to the electrical stimulation generation circuitry. In another step, a first electrical stimulation is provided from the stimulation generation circuitry to the electrode. A second indication may be generated, which is indicative of the first stimulation is being provided to the stimulating tip and is further indicative of the first stimulation being prevented from being received by the device through a return electrode. While the method may end here, it is preferred to again apply the first electrical stimulation to a targeted animal tissue region and generate a third indication, which is indicative of the first stimulation being received by the device through the return electrode.

According to another aspect of the present invention, all of the indications are generated within a three-dimensional surgical field including the targeted tissue region. One or more of the indications may be visual or audio indications. Visual indications may be generated by a visual indicator disposed between the stimulating tip and all user operable controls.

According to an aspect of a visual indicator according to the present invention, the visual indicator may include a light ring having a proximal surface and a distal surface coupled by an oblique annular surface. Either or both of the proximal and/or distal surfaces may include a circular perimeter. In a preferred configuration, the distal perimeter is smaller than the proximal perimeter. Light may be transmitted through the light ring annular surface, distally away from the electrode. A visual indicator according to the present invention may further include a reflective element mounted adjacent the proximal surface of the light ring.

In another embodiment of a method according to the present invention, a method of applying electrical stimulation to a targeted animal tissue region includes the step of receiving, into a single human hand, a device. The device includes a housing extending along a housing longitudinal axis between a housing proximal end and a housing distal end, electrical stimulation generation circuitry at least substantially contained within the housing, and one or more user operable controls coupled to the housing and in operational communication with the electrical stimulation generation circuitry. Extending from the proximal end of the housing is an operative element which includes an electrode operatively coupled to the electrical stimulation generation circuitry. Also contained at least substantially within the housing is a power supply that is electrically coupled to the electrical stimulation generation circuitry. The housing may further include a gripping portion comprising a location channel extending longitudinally distally from one of the user operable controls. The method may include using a finger of the hand, preferably the index finger, to identify the location channel. One of the user operable controls which is proximal the location channel may be located and then manipulated. The user operable control may be located by sliding the finger proximally along the location channel. All of the steps of this method may be performed within a three-dimensional surgical field. Alternatively, one or more of the steps, such as the step of receiving the device, may be performed outside of a surgical field.

According to an aspect of a relationship between the operative element and the location channel provided on the device, the operative element may include an angled or curvilinear portion, thereby defining an operative element plane. The location channel and/or the located user operable control may be disposed substantially coplanar with the operative element plane.

Features and advantages of the inventions are set forth in the following Description and Drawings, as well as the appended description of technical features.

### BRIEF DESCRIPTION OF THE DRAWINGS

FIG. 1 is a diagrammatic view of a system usable in association with a family of different monitoring and treatment devices for use in different medical procedures.

FIG. 2 is a perspective view showing an exemplary embodiment of the system shown in FIG. 1, the stimulation control device being removably coupled to a stimulation probe, and showing the stimulation signal path through the system.

FIG. 3A is a side view with a portion broken away and in section showing the stimulation probe having the stimulation control device embedded within the stimulation probe.

FIG. 3B is a side view with a portion broken away and in section showing the stimulation probe having the stimulation control device embedded within the stimulation probe, and showing an optional needle-like return electrode.

FIG. 3C is a side view with a portion broken away and in section showing an additional embodiment of the stimulation probe having a housing that includes a gripping base and a flexible nose cone, and an illuminating ring indicator.

FIG. 4A is a side view of the stimulation probe of FIG. 3c, showing the users hand in a position on the stimulation probe to move the flexible nose cone.

FIG. 4B is a side view of the stimulation probe of FIG. 4A, showing the users hand flexing the flexible nose cone.

FIG. 5 is a side view with a portion broken away and in section showing elements of the flexible nose cone, the ring indicator, and the gripping base.

FIG. 6 is a graphical view of a desirable biphasic stimulus pulse output of the stimulation device.

FIG. 7 is a view showing how the geometry of the stimulation control device shown in FIG. 2 aids in its positioning during a surgical procedure.

FIG. 8 is a block diagram of a circuit that the stimulation control device shown throughout the FIGS. can incorporate.

FIGS. 9A and 9B are perspective views showing the stimulation control device in use with a cutting device.

FIGS. 10A and 10B are perspective views showing the stimulation control device in use with a drilling or screwing device.

FIGS. 11A and 11B are perspective views showing the stimulation control device in use with a pilot auger device.

FIGS. 12A and 12B are perspective views showing the stimulation control device in use with a fixation device.

FIG. 13 is a plane view of a kit used in conjunction with the stimulation probe shown in FIG. 3C, and including the stimulation probe and instructions for use.

FIG. 14 is a perspective view of the stimulation probe shown in FIG. 3C.

FIG. 15 is an exploded view of the stimulation probe shown in FIG. 14.

FIG. 16 is a side elevation view of a light permeable ring which may be used as a part of a visual indicator according to the present invention.

FIG. 17 is a distal elevation view of the ring of FIG. 16.

FIG. 18 is a partial assembly view of an embodiment of a device according to the present invention using the ring of FIG. 16.

FIG. 19 is a perspective view of an embodiment of a device according to the present invention depicting a relationship between a location channel, a user operable control, a visual indicator, and a user operable element.

The invention may be embodied in several forms without departing from its spirit or essential characteristics. The scope of the invention is defined in the appended claims, rather than in the specific description preceding them. All embodiments that fall within the meaning and range of equivalency of the claims are therefore intended to be embraced by the claims.

### DESCRIPTION OF PREFERRED EMBODIMENTS

This Specification discloses various systems and methods for safeguarding against nerve, muscle, and tendon injury during surgical procedures or confirming the identity and/or location of nerves, muscles, and tendons and evaluating their function or the function of muscles enervated by those nerves. The systems and methods are particularly well suited for assisting surgeons in identification of nerves and muscles in order to assure nerve and muscle integrity during medical procedures using medical devices such as stimulation monitors, cutting, drilling, and screwing devices, pilot augers, and fixation devices. For this reason, the systems and methods will be described in the context of these medical devices.

The systems and methods desirably allow the application of a stimulation signal at sufficiently high levels for the purposes of locating, stimulating, and evaluating nerve or muscle, or both nerve and muscle integrity in numerous medical procedures, including, but not limited to, evaluating proximity to a targeted tissue region, evaluating proximity to a nerve or to identify nerve tissue, evaluating if a nerve is intact (i.e., following a traumatic injury) to determine if a repair may be needed, evaluating muscle contraction to determine whether or not the muscle is innervated and/or whether the muscle is intact and/or whether the muscle is severed, and evaluating muscle and tendon length and function following a repair or tendon transfer prior to completing a surgical procedure.

Still, it should be appreciated that the disclosed systems and methods are applicable for use in a wide variety of medical procedures with a wide variety of medical devices. By way of non-limiting example, the various aspects of the invention have application in procedures requiring grasping medical devices and internal viewing devices as well.

### I. Overview of the System

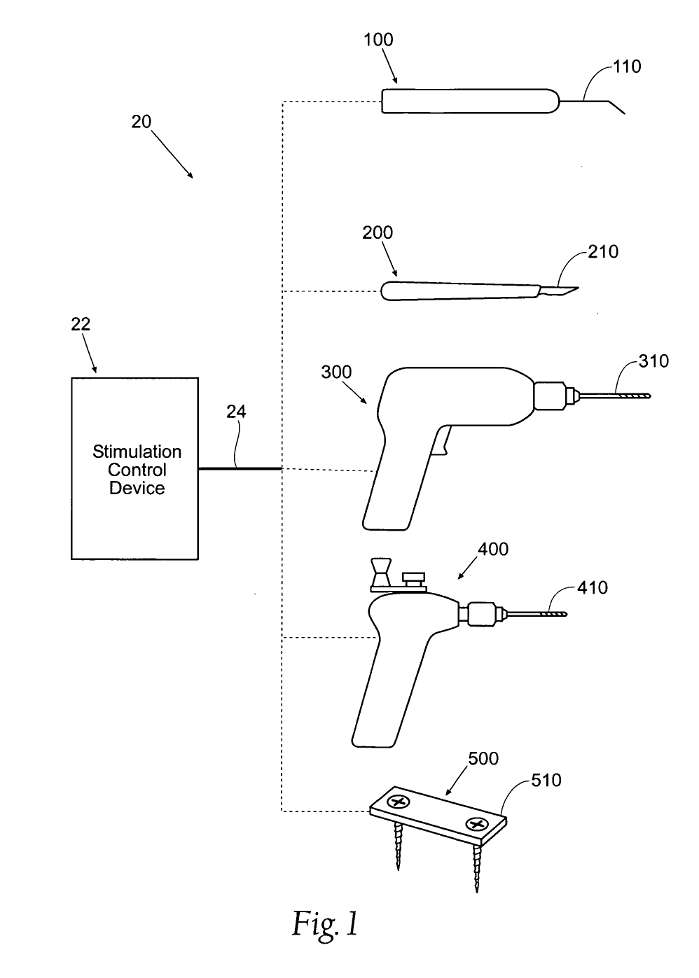

FIG. 1 shows an illustrative system 20 for locating and identifying tissue and safeguarding against tissue and/or bone injury during surgical procedures. In the illustrated embodiment, the system 20 is configured for locating, monitoring, and stimulating tissue and other structures throughout the body. The system 20 includes a stimulation control device 22 operating individually or in conjunction with one or more of a family of stimulating medical devices including, for example, a stimulation monitor or probe 100, a cutting device 200, a drilling or screwing device 300, a pilot auger 400, and a fixation device 500.

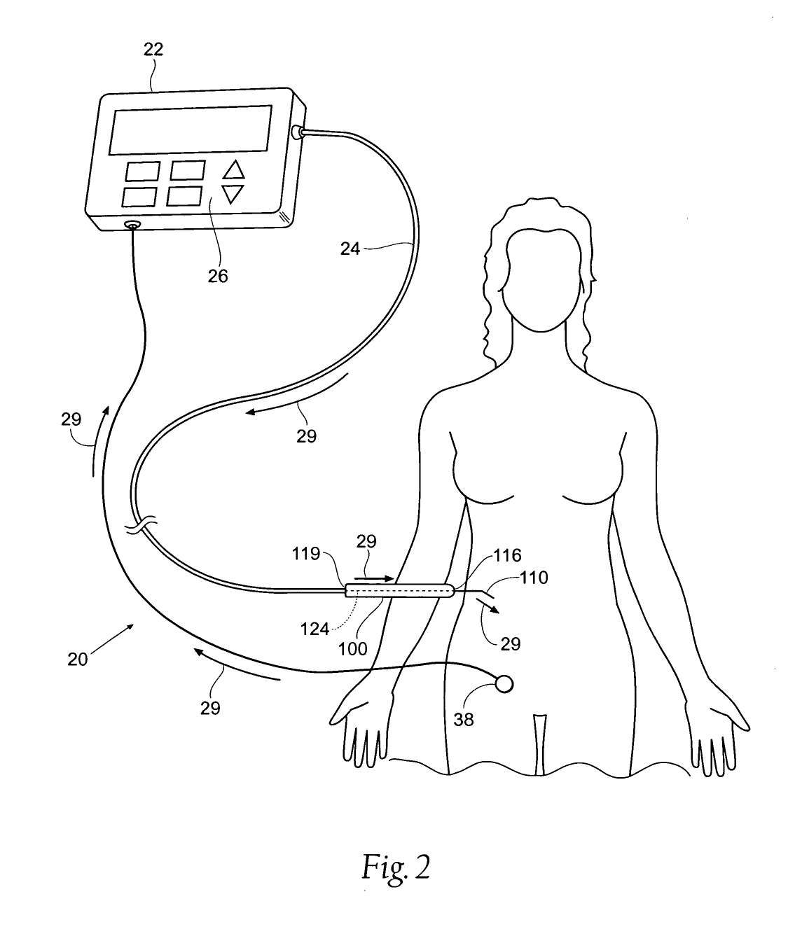

In an exemplary embodiment, and as can be seen in FIG. 2, the stimulation control device 22 functions in the system 20 to generate an electrical stimulation signal 29. The stimulation signal 29 flows from the stimulation control device 22 through a lead 24 to a medical device (e.g., stimulation probe 100). The stimulation signal 29 then flows through a predefined insulated path 124 within the stimulation probe 100 and to an operative element, such as an electrically conductive surface, i.e., a coupled electrode 110. The electrode 110 is to be positioned on or near a region of a patient to be stimulated. In monopolar operation, a return electrode (or indifferent electrode) 38 provides an electrical path from the body back to the control device 22. The stimulation control device 22 may operate in a monopolar or bipolar configuration, as will be described in greater detail later.

The stimulation signal 29 is adapted to provide an indication or status of the device. The indication may include a physical motor response (e.g., twitching), and/or one or more visual or audio signals from the stimulation control device 22, which indicate to the surgeon the status of the device, and/or close proximity of the electrode 110 to a nerve, or a muscle, or a nerve and a muscle. The stimulation control device may also indicate to the surgeon that the stimulation control device is operating properly and delivering a stimulus current.

### II. Medical Devices

The configuration of the stimulating medical devices that form a part of the system can vary in form and function. Various representative embodiments of illustrative medical devices will be described.

A. Stimulation Probe

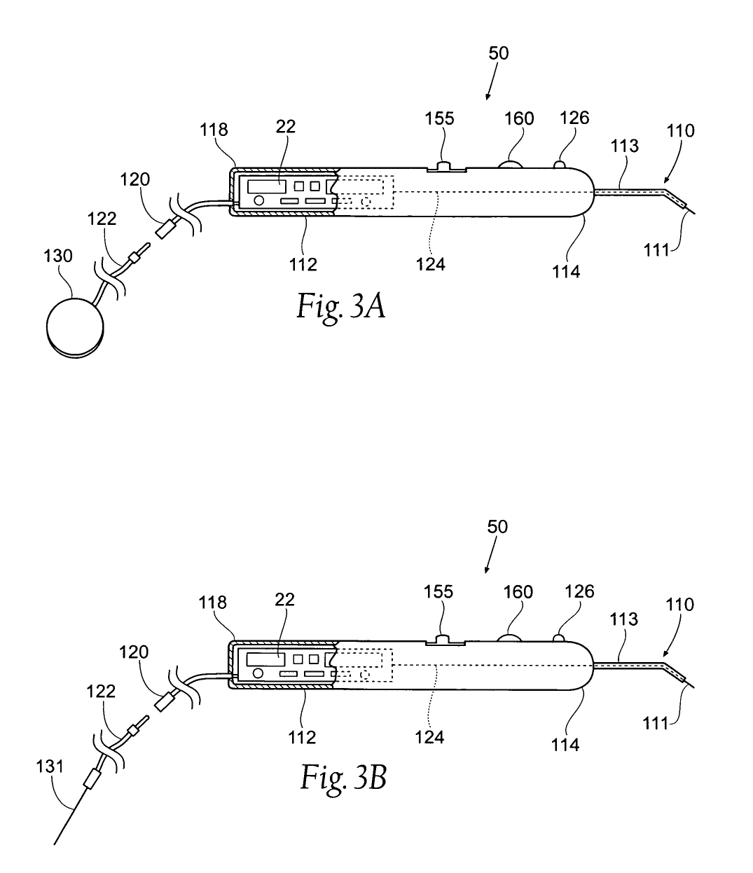

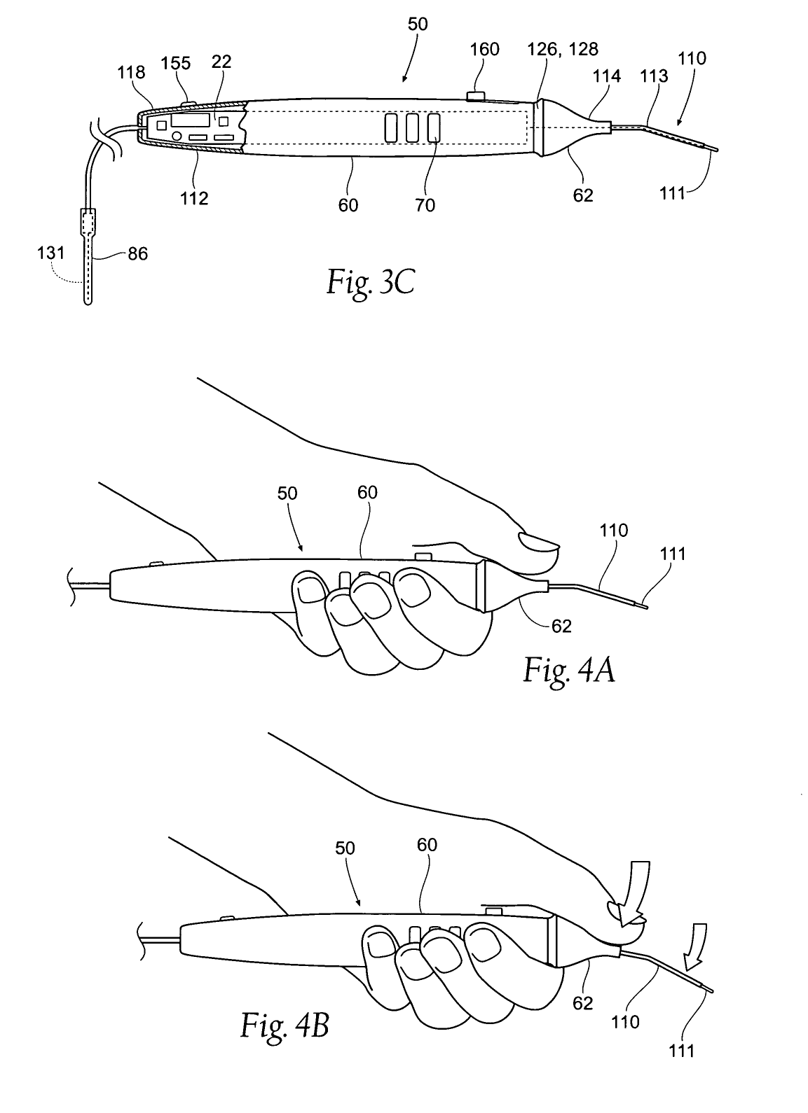

FIGS. 3A to 3C show various embodiments of a hand held stimulation monitor or probe 50 for identification and testing of nerves and/or muscles during surgical procedures. As shown, the stimulation probe 50 may accommodate within a generally tubularly housing 112 the electrical circuitry of a stimulation control device 22. The stimulation probe 50 is desirably an ergonomic, sterile, single use instrument intended for use during surgical procedures to identify nerves and muscles, muscle attachments, or to contract muscles to assess the quality of surgical interventions or the need for surgical interventions, or to evaluate the function of nerves already identified through visual means. The stimulation probe 50 may be sterilized using ethylene oxide, for example.

The stimulation probe 50 is preferably sized small enough to be held and used by one hand during surgical procedures, and is ergonomically designed for use in either the left or right hand. In a representative embodiment, the stimulation probe 50 may have a width of about 20 millimeters to about 30 millimeters, and desirably about 25 millimeters. The length of the stimulation probe 50 (not including the operative element 110) may be about 18 centimeters to about 22 centimeters, and desirably about 20 centimeters. The operative element 110 may also include an angle or bend to facilitate access to deep as well as superficial structures without the need for a large incision. The operative element 110 will be described in greater detail later. A visual or audio indicator 126 incorporated with the housing 112 provides reliable feedback to the surgeon as to the request and delivery of stimulus current.

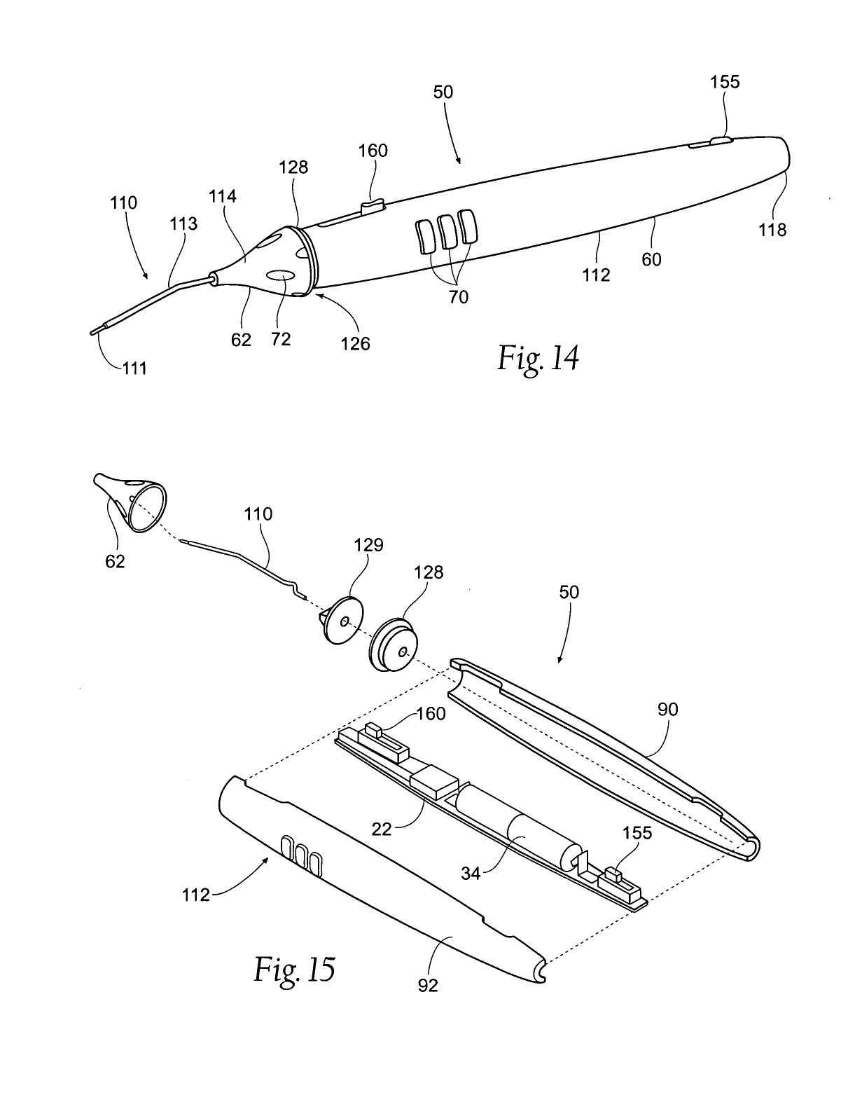

In one embodiment shown in FIGS. 3C and 14, the stimulation probe 50 includes a housing 112 that comprises a gripping base portion 60 and an operative element adjustment portion 62. The operative element 110 extends from the proximal end of the adjustment portion 62. In order to aid the surgeon in the placement of the operative element 110 at the targeted tissue region, the adjustment portion, as will be described as a nose cone 62, may be flexible. This flexibility allows the surgeon to use either a finger or a thumb positioned on the nose cone 62 to make fine adjustments to the position of stimulating tip 111 of the operative element 110 at the targeted tissue region (see FIGS. 4A and 4B). The surgeon is able to grasp the gripping base 60 with the fingers and palm of the hand, and position the thumb on the nose cone 62, and with pressure applied with the thumb, cause the stimulating tip 111 to move while maintaining a steady position of the gripping base portion 62. This flexible nose cone 62 feature allows precise control of the position of the stimulating tip 111 with only the movement of the surgeon's thumb (or finger, depending on how the stimulating probe is held).

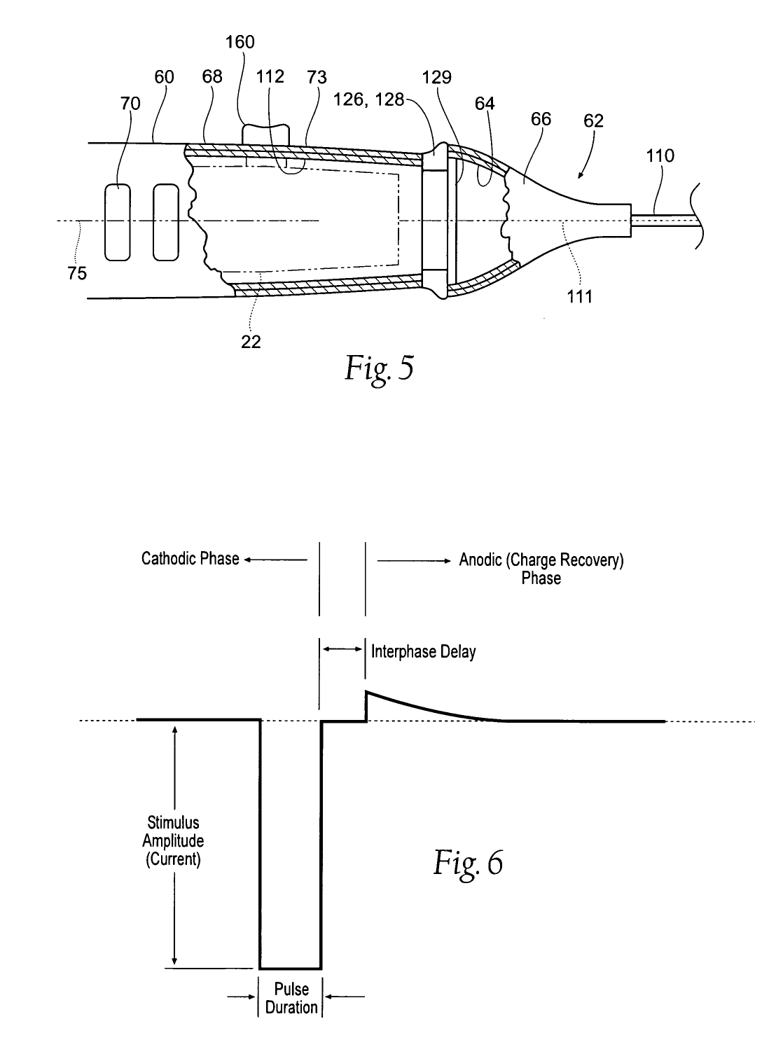

The flexible nose cone 62 may comprise a single element or it may comprise at least an inner portion 64 and an outer portion 66, as shown in FIG. 5. In order to facilitate some flexibility of the proximal portion 114 of the stimulation probe 50, the inner portion 64 of the nose cone 62 may be made of a thermoplastic material having some flexibility. One example may be LUSTRAN® ABS 348, or similar material. The outer portion 66 may comprise a softer over molded portion and may be made of a thermoplastic elastomer material having some flexibility. One example may be VERSAFLEX™ OM 3060-1 from GLS Corp. The nose cone 62 is desirably generally tapered. For example, the nose cone 62 may be rounded, as shown in FIGS. 3A and 3B, or the nose cone may be more conical in shape, as shown in FIG. 3C.

The nose cone 62 may also include one or more features, such as ribs or dimples 72, as shown in FIG. 14, to improve the gripping, control, and stability of the stimulation probe 50 within the surgeon's hand.

The gripping base portion 60 of the housing 112 may also include an overmolded portion 68. The overmolded portion 68 may comprise the full length of the gripping base portion 60, or only a portion of the gripping base 60. The soft overmolded portion 68 may include one or more features, such as dimples or ribs 70, as shown, to improve the gripping, control, and stability of the stimulation probe 50 within the surgeon's hand. The overmolded portion 68 may comprise the same or similar material as the thermoplastic elastomer material used for the outer portion 66 of the flexible nose cone 62.

In one embodiment, the stimulation probe 50 includes a housing 112 that carries an insulated lead 124. The insulated lead 124 connects the operative element 110 positioned at the housing's proximal end 114 to the circuitry 22 within the housing 112 (see FIG. 3A). It is to be appreciated that the insulated lead is not necessary and the operative element 110 may be coupled to the circuitry 22 (see FIG. 3C). The lead 124 within the housing 112 is insulated from the housing 112 using common insulating means (e.g., wire insulation, washers, gaskets, spacers, bushings, and the like). The conductive tip 111 of the operative element 110 is positioned in electrical conductive contact with at least one muscle, or at least one nerve, or at least one muscle and nerve.

As shown, the stimulation probe 50 is mono-polar and is equipped with a single operative element (i.e., electrode) 110 at the housing proximal end 114. A return electrode 130, 131 may be coupled to the stimulation probe 50 and may be any of a variety of electrode types (e.g., paddle, needle, wire, or surface), depending on the surgical procedure being performed. As shown, the various return electrodes 130, 131 are coupled to the housing distal end 118. In an alternative embodiment, the stimulation device 50 itself may be bipolar by including a return electrode in the operative element 110, which precludes the use of a return electrode coupled to the stimulation probe 50.

As shown and described, the stimulation probe 50 may accommodate within the housing 112 the electrical circuitry of a stimulation control device 22. In this arrangement, the stimulation probe 50 may have one or more user operable controls. Two are shown—155 and 160. Power switch 155 serves a dual purpose of turning the stimulation probe 500N and OFF (or standby), and also can be stepped to control the stimulation signal amplitude selection within a predefined range (e.g., 0.5, 2.0, and 20 mA). In this configuration, the switch may be a four position switch. Before the first use of the stimulation probe 50, the power switch 155 is in the OFF position and keeps the stimulation probe off. After the stimulation probe 50 has been turned ON—by moving the switch 155 to an amplitude selection—the OFF position now corresponds to a standby condition, where no stimulation would be delivered. In one embodiment, once the stimulation probe 50 has been turned on, it cannot be turned off, it can only be returned to the standby condition and will remain operational for a predetermined time, e.g., at least about seven hours. This feature is intended to allow the stimulation probe 50 to only be a single use device, so it can not be turned OFF and then used again at a later date.

The pulse control device 160 allows for adjustment of the stimulation signal pulse width from a predefined range (e.g., about zero to about 200 microseconds). In one embodiment, the pulse control 160 may be a potentiometer to allow a slide control to increase or decrease the stimulation signal pulse width within the predefined range.

The stimulation pulse may have a non-adjustable frequency in the range of about 10 Hz to about 20 Hz, and desirably about 16 Hz.

As a representative example, the stimulation pulse desirably has a biphasic waveform with controlled current during the cathodic (leading) phase, and net DC current less than 10 microamps, switch adjustable from about 0.5 milliamps to about 20 milliamps, and pulse durations adjustable from about zero microseconds up to about 200 microseconds. A typical, biphasic stimulus pulse is shown in FIG. 6.

The operative element 110 exits the housing 112 at the proximal end 114 to deliver stimulus current to the excitable tissue. The operative element 110 comprises a length and a diameter of a conductive material, and is desirably fully insulated with the exception of the most proximal end, e.g. about 1.0 millimeters to about 10 millimeters, and desirably about 4 millimeters to about 6 millimeters, which is non-insulated and serves as the stimulating tip or surface (or also referred to as active electrode) 111 to allow the surgeon to deliver the stimulus current only to the intended tissue. The small area of the stimulating surface 111 (the active electrode) of the operative element 110 ensures a high current density that will stimulate nearby excitable tissue. The insulation material 113 may comprise a medical grade heat shrink.

The conductive material of the operative element 110 comprises a diameter having a range between about 0.5 millimeters to about 1.5 millimeters, and may be desirably about 1.0 millimeters. The length of the operative element 110 may be about 50 millimeters to about 60 millimeters, although it is to be appreciated that the length may vary depending on the particular application. As shown, the operative element 110 may include one or more bends to facilitate accurate placement of the stimulating surface 111. In one embodiment, the conductive-material of operative element 110 is made of a stainless steel 304 solid wire, although other known conductive materials may be used.

As previously described, in monopolar operation, a return electrode (or indifferent electrode) 130 or 131, for example, provides an electrical path from the body back to the control device 22 within the housing 112. The return electrode 130 (see FIG. 3A) may be placed on the surface of intact skin (e.g., surface electrodes as used for ECG monitoring during surgical procedures) or it might be needle-like 131 (see FIGS. 3B and 3C), and be placed in the surgical field or penetrate through intact skin. The housing's distal end 118 can incorporate a connector or jack 120 which provides options for return current pathways, such as through a surface electrode 130 or a needle electrode 131, having an associated plug 122. It is to be appreciated that a return electrode and associated lead may be an integral part of the stimulation probe 50, i.e., no plug or connector, as shown in FIG. 3C.

Additionally, the device 50 may desirably incorporate a visual or audio indicator 126 for the surgeon. This visual or audio indicator 126 allows the surgeon to confirm that the stimulator 50 is delivering stimulus current to the tissue it is contacting. Through the use of different tones, colors, different flash rates, etc., the indicator 126 (which can take the form, e.g., of a light emitting diode (LED)) allows the surgeon to confirm that the stimulating tip 111 is in place, the instrument is turned ON, and that stimulus current is flowing. Thus the surgeon has a much greater confidence that the failure to elicit a muscle contraction is because of lack of viable nervous tissue near the tip 111 of the stimulator 50 rather than the failure of the return electrode connection or some other instrumentation problem.

As a representative example, in use the indicator 126 may be configured to illuminate continuously in one color when the stimulation probe 50 is turned on but not in contact with tissue. After contact with tissue is made, the indicator 126 may flash (i.e., blink) to indicate that stimulation is being delivered. If the stimulation has been requested, i.e., the stimulation probe has been turned on, but there is no stimulation being delivered because of a lack of continuity between the operative element 110 and the return electrode 130, or an inadequate connection of the operative element 110 or the return electrode 130 to the patient tissue, the indicator 126 may illuminate in a different color, and may illuminate continuously or may flash.

In one embodiment, as can be best seen in FIGS. 3C and 5, the indicator 126 comprises a ring indicator 128 that provides a visual indication around at least a portion, and desirably all of the circumference of the stimulation probe 50 generally near the flexible nose cone 62. The visual ring indicator 128 may be an element of the gripping portion 60, or it may be an element of the flexible nose cone 62, or the ring indicator may positioned between the gripping portion 60 and the flexible nose cone 62. The ring indicator 128 may also include a reflective element 129 to improve and focus the illumination effect of the light emitting source, e.g., one or more LEDs. The ring indicator 128 and the reflective element may be a single component, or more than one component (as can be seen in FIGS. 5 and 15).

Audio feedback also makes possible the feature of assisting the surgeon with monitoring nerve integrity during surgery. The insulated lead 124 connects to the operative element 110 that, in use, is positioned within the surgical field on a nerve distal to the surgical site. Stimulation of the nerve causes muscle contraction distally. The stimulation control device 22 incorporated within the housing 112 may be programmed to provide an audio tone followed by a stimulation pulse at prescribed intervals. The audio tone reminds the surgeon to observe the distal muscle contraction to confirm upon stimulation that the nerve is functioning and intact.

FIG. 15 shows an exploded view of a representative stimulation probe 50. As can be seen, the stimulation control device 22 is positioned within the housing 112. A battery 34 is electrically coupled to the control device 22. A first housing element 90 and a second housing element 92 partially encapsulate the control device 22. The ring indicator 128 and the reflective element 129 are coupled to the proximal end of the housing 112. The operative element 110 extends through the nose cone 62 and couples to the control device 22. Desirably, the stimulation probe 50 will be constructed in a manner to conform to at least the IPX1 standard for water ingress.

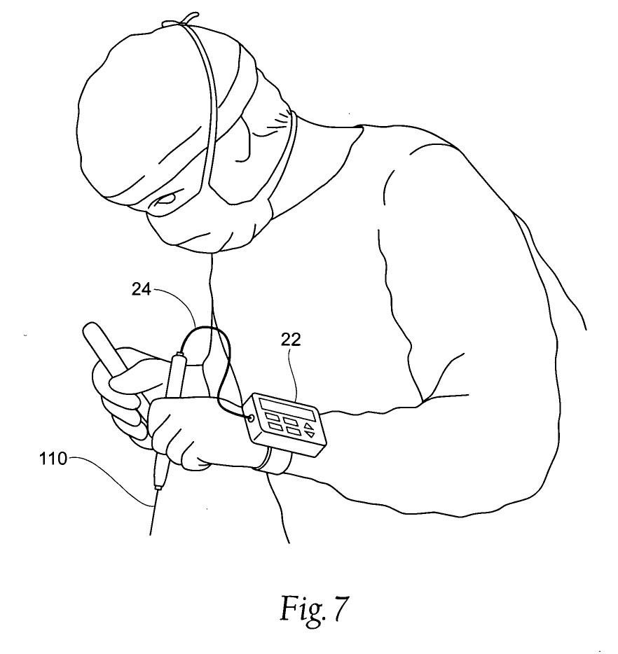

Alternatively, as FIG. 2 shows, the stimulation control device 22 may be housed in a separate case, with its own input/output (I/O) controls 26. In this alternative arrangement, the stimulation control device 22 is sized small enough to be easily removably fastened to a surgeon's arm or wrist during the surgical procedure, or otherwise positioned in close proximity to the surgical location (as shown in FIG. 7), to provide sufficient audio and/or visual feedback to the surgeon. In this arrangement, the separate stimulation control device 22 can be temporarily coupled by a lead to a family of various medical devices for use.

The present invention includes a method of identifying/locating tissue, e.g., a nerve or muscle, in a patient that comprises the steps of providing a hand-held stimulation probe 50, 100 as set forth above, engaging a patient with the first operative element 110 and the second electrode 130, moving the power switch 155 to an activation position causing a stimulation signal 29 to be generated by the stimulation control device 22 and transmitted to the first operative element 110, through the patient's body to the second electrode 130, and back to the stimulation control device 22. The method may also include the step of observing the indicator 126 to confirm the stimulation probe 50, 100 is generating a stimulation signal. The method may also include the step of observing a tissue region to observe tissue movement or a lack thereof.

B. The Stimulation Control Device

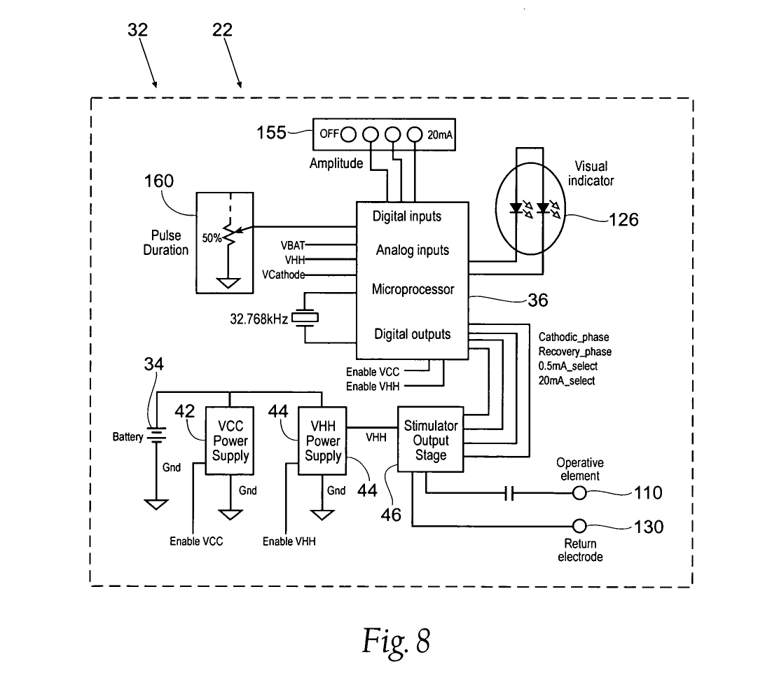

As FIG. 8 shows, the stimulation control device 22 includes a circuit 32 that generates electrical stimulation waveforms. A battery 34 desirably provides the power. The control device 22 also desirably includes an on-board, programmable microprocessor 36, which carries embedded code. The code expresses pre-programmed rules or algorithms for generating the desired electrical stimulation waveforms using the stimulus output circuit 46 and for operating the visible or audible indicator 126 based on the controls actuated by the surgeon.

In one form, the size and configuration of the stimulation control device 22 makes for an inexpensive device, which is without manual internal circuit adjustments. It is likely that the stimulation control device 22 of this type will be fabricated using automated circuit board assembly equipment and methods.

C. Incorporation with Surgical Devices

A stimulation control device 22 as just described may be electrically coupled through a lead, or embedded within various devices commonly used in surgical procedures (as previously described for the stimulation probe 50).

1. Cutting Device

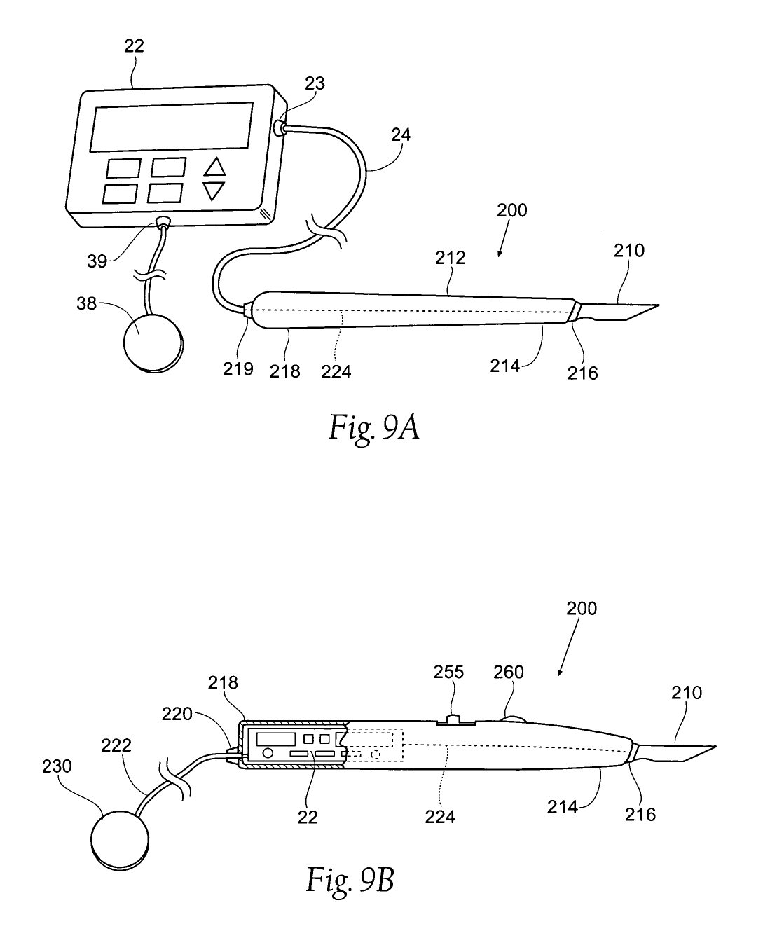

In FIGS. 9A and 9B, a device 200 is shown that incorporates all the features disclosed in the description of the stimulation probe 50, 100, except the device 200 comprises the additional feature of providing an “energized” surgical device or tool. FIG. 9A shows the tool to be a cutting device 200 (e.g., scalpel) removably coupled to a stimulation control device 22.

In the embodiment shown, the cutting device 200 includes a body 212 that carries an insulated lead 224. The insulated lead 224 connects to an operative element, such as electrode 210, positioned at the body proximal end 214 and a plug-in receptacle 219 at the body distal end 118. The lead 224 within the body 212 is insulated from the body 212 using common insulating means (e.g., wire insulation, washers, gaskets, spacers, bushings, and the like).

In this embodiment, the electrode 210 performs the cutting feature (e.g., knife or razor). The electrode 210 performs the cutting feature in electrical conductive contact with at least one muscle, or at least one nerve, or at least one muscle and nerve. The cutting device 200 desirably includes a plug-in receptacle 216 for the electrode 210, allowing for use of a variety of cutting electrode shapes and types (e.g., knife, razor, pointed, blunt, curved), depending on the specific surgical procedure being performed. In this configuration, the lead 224 electrically connects the electrode 210 to the stimulation control device 22 through plug-in receptacle 219 and lead 24.

In one embodiment, the cutting device 200 is mono-polar and is equipped with a single electrode 210 at the body proximal end 214. In the mono-polar mode, the stimulation control device 22 includes a return electrode 38 which functions as a return path for the stimulation signal. Electrode 38 may be any of a variety of electrode types (e.g., paddle, needle, wire, or surface), depending on the surgical procedure being performed. The return electrode 38 may be attached to the stimulation device 22 by way of a connector or plug-in receptacle 39. In an alternative embodiment, the cutting device 200 may be bipolar, which precludes the use of the return electrode 38.

In the embodiment shown in FIG. 9B, the cutting device 200 accommodates within the body 212 the electrical circuitry of the stimulation control device 22. In this arrangement, the cutting device 200 may have at least two operational slide controls, 255 and 260.

Power switch 255 serves a dual purpose of turning the stimulation signal to the cutting device 200 on and off, and also is stepped to control the stimulation signal amplitude selection from a predefined range (e.g., 0.5, 2.0, and 20 mA). The pulse control switch 260 allows for adjustment of the stimulation signal pulse width from a predefined range (e.g., zero through 200 microseconds).

At the body distal end 218, a second plug-in receptacle 220 may be positioned for receipt of a second lead 222. Lead 222 connects to electrode 230 which functions as a return path for the stimulation signal when the cutting device 200 is operated in a mono-polar mode.

Additionally, the device 200 may incorporate a visual or audio indicator for the surgeon, as previously described.

The present invention includes a method of identifying/locating tissue, e.g., a nerve or muscle, in a patient that comprises the steps of providing cutting device 200 as set forth above, engaging a patient with the first electrode 210 and the second electrode 230, moving the power switch 255 to an activation position causing a stimulation signal 29 to be generated by the stimulation control device 22 and transmitted to the first electrode 210, through the patient's body to the second electrode 230, and back to the stimulation control device 22. The method may also include the step of observing the indicator 126 to confirm the cutting device 200 is generating a stimulation signal. The method may also include the step of observing a tissue region to observe tissue movement or a lack thereof.

2. Drilling Device

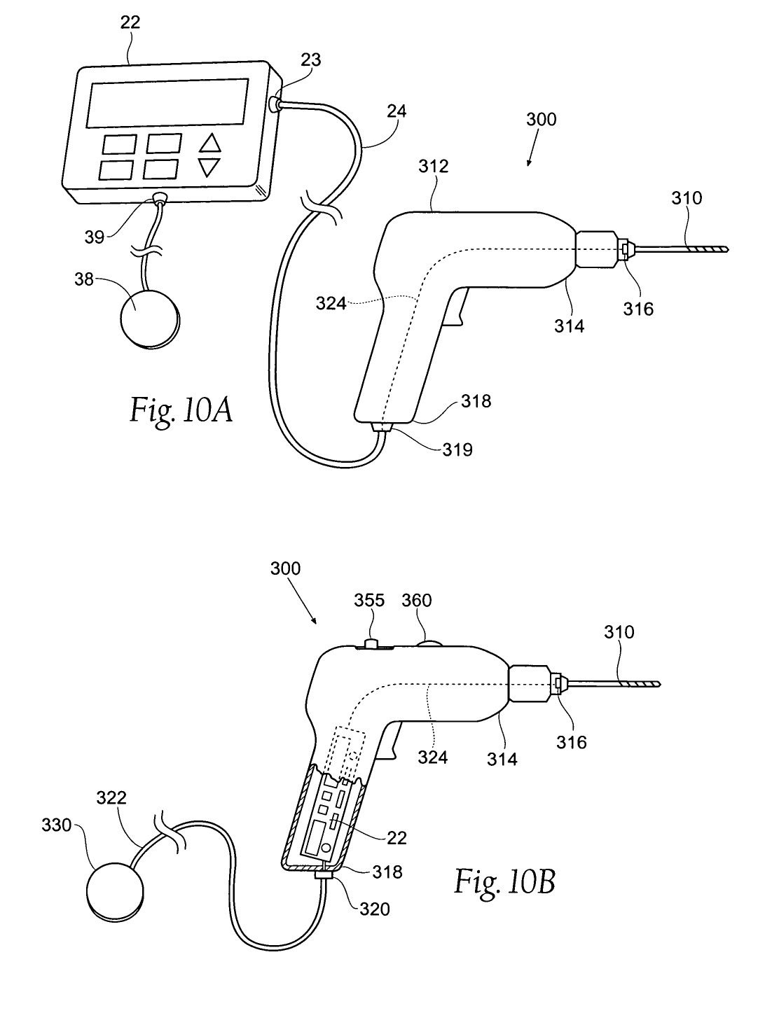

In FIGS. 10A and 10B, a device 300 is shown that incorporates all the features disclosed in the description of the stimulation probe 50, 100, except the device 300 comprises the additional feature of providing an “energized” surgical device or tool, which comprises a drilling device 300. In FIG. 10A is drilling device 300 is removably coupled to a stimulation control device 22.

In the embodiment shown, the drilling device 300 includes a body 312 that carries an insulated lead 324. The insulated lead 324 connects to an operative element, such as electrode 310, positioned at the body proximal end 314 and a plug-in receptacle 319 at the body distal end 318. The lead 324 within the body 312 is insulated from the body 312 using common insulating means (e.g., wire insulation, washers, gaskets, spacers, bushings, and the like).

In this embodiment, the electrode 310 performs the drilling feature. The electrode 310 may also perform a screwing feature as well. The electrode 310 performs the drilling feature in electrical conductive contact with a hard structure (e.g., bone).

The drilling device 300 desirably includes a plug-in receptacle or chuck 316 for the electrode 310, allowing for use of a variety of drilling and screwing electrode shapes and sizes (e.g., ¼ and ⅜ inch drill bits, Phillips and flat slot screw drivers), depending on the specific surgical procedure being performed. In this configuration, the lead 324 electrically connects the electrode 310 to the stimulation control device 22 through plug-in receptacle 319 and lead 324.

In one embodiment, the drilling device 300 is mono-polar and is equipped with a single electrode 310 at the body proximal end 314. In the mono-polar mode, the stimulation control device 22 includes a return electrode 38 which functions as a return path for the stimulation signal. Electrode 38 may be any of a variety of electrode types (e.g., paddle, needle, wire, or surface), depending on the surgical procedure being performed. The return electrode 38 may be attached to the stimulation device 22 by way of a connector or plug-in receptacle 39. In an alternative embodiment, the drilling device 300 may be bipolar, which precludes the use of the return electrode 38.

In FIG. 10B, the drilling device 300 is shown to accommodate within the body 312 the electrical circuitry of the stimulation control device 22. The drilling device 300 may have at least two operational slide controls, 355 and 360. Power switch 355 serves a dual purpose of turning the stimulation signal to the drilling device 300 on and off, and also is also stepped to control the stimulation signal amplitude selection from a predefined range (e.g., 0.5, 2.0, and 20 mA). The pulse control switch 360 allows for adjustment of the stimulation signal pulse width from a predefined range (e.g., zero through 200 microseconds). At the body distal end 318, a second plug-in receptacle 320 may be positioned for receipt of a second lead 322. Lead 322 connects to electrode 330 which functions as a return path for the stimulation signal when the drilling device 300 is operated in a mono-polar mode.

Additionally, the device 300 may incorporate a visual or audio indicator for the surgeon, as previously described.

The present invention includes a method of identifying/locating tissue, e.g., a nerve or muscle, in a patient that comprises the steps of providing a drilling device 300 as set forth above, engaging a patient with the first electrode 310 and the second electrode 330, moving the power switch 355 to an activation position causing a stimulation signal 29 to be generated by the stimulation control device 22 and transmitted to the first electrode 310, through the patient's body to the second electrode 330, and back to the stimulation control device 22. The method may also include the step of observing the indicator 126 to confirm the drilling device 400 is generating a stimulation signal. The method may also include the step of observing a tissue region to observe tissue movement or a lack thereof.

3. Pilot Auger

An additional aspect of the invention provides systems and methods for controlling operation of a family of stimulating devices comprising a stimulation control device electrically coupled to a pilot auger for hard surface rotary probing.

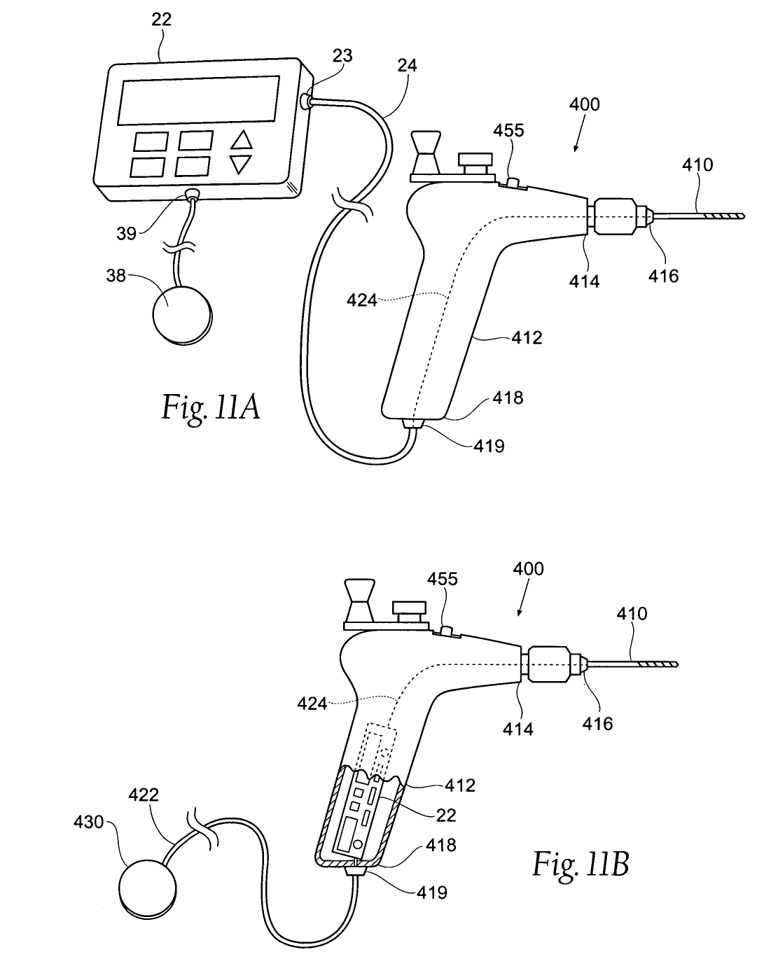

This embodiment incorporates all the features disclosed in the description of the stimulation probe 50, 100, except this embodiment comprises the additional feature of providing an “energized” surgical device or tool. FIG. 11A shows a pilot auger device 400 removably coupled to a stimulation control device 22. In the embodiment shown, the pilot auger device 400 includes a body 412 that carries an insulated lead 424. The insulated lead 424 connects to an operative element, such as an electrode 410, positioned at the body proximal end 414 and a plug-in receptacle 419 at the body distal end 418. The lead 424 within the body 412 is insulated from the body 412 using common insulating means (e.g., wire insulation, washers, gaskets, spacers, bushings, and the like). In this embodiment, the electrode 410 performs the pilot augering feature. The electrode 410 performs the pilot augering feature in electrical conductive contact with a hard structure (e.g., bone).

The pilot auger device 400 desirably includes a plug-in receptacle or chuck 416 for the electrode 410, allowing for use of a variety of pilot augering electrode shapes and sizes (e.g., 1/32, 1/16,and ⅛ inch), depending on the specific surgical procedure being performed. In this configuration, the lead 24 electrically connects the electrode 410 to the stimulation control device 22 through plug-in receptacle 419 and lead 24.

In one embodiment, the pilot auger device 400 is mono-polar and is equipped with a single electrode 410 at the body proximal end 414. In the mono-polar mode, the stimulation control device 22 includes a return electrode 38 which functions as a return path for the stimulation signal. Electrode 38 may be any of a variety of electrode types (e.g., paddle, needle, wire, or surface), depending on the surgical procedure being performed. The return electrode 38 may be attached to the stimulation device 22 by way of a connector or plug-in receptacle 39. In an alternative embodiment, the pilot auger device 400 may be bipolar, which precludes the use of the return electrode 38.

As FIG. 11B shows. the pilot auger device 400 may accommodate within the body 412 the electrical circuitry of the stimulation control device 22. At the body distal end 418, a second plug-in receptacle 420 may be positioned for receipt of a second lead 422. Lead 422 connects to electrode 430 which functions as a return path for the stimulation signal when the pilot auger device 400 is operated in a mono-polar mode.

The pilot auger device 400 includes a power switch 455. When moved to an activation position, a stimulation signal is generated by the stimulation control device 22. Additionally, the device 400 may incorporate a visual or audio indicator for the surgeon, as previously described.

The present invention includes a method of identifying/locating tissue, e.g., a nerve or muscle, in a patient that comprises the steps of providing a pilot auger device 400 as set forth above, engaging a patient with the first electrode 410 and the second electrode 430, moving the power switch 455 to an activation position causing a stimulation signal to be generated by the stimulation control device 22 and transmitted to the first electrode 410, through the patient's body to the second electrode 430, and back to the stimulation control device 22. The method may also include the step of observing the indicator 126 to confirm the pilot auger device 400 is generating a stimulation signal. The method may also include the step of observing a tissue region to observe tissue movement or a lack thereof.

D. Incorporation with Fixation Devices

An additional aspect of the invention provides systems and methods for controlling operation of a family of stimulating devices comprising a stimulation control device electrically coupled to a fixation device or a wrench or screwdriver for placing the fixation device. A fixation device (e.g., orthopedic hardware, pedicle screws) is commonly used during spinal stabilization procedures (fusion), and internal bone fixation procedures.

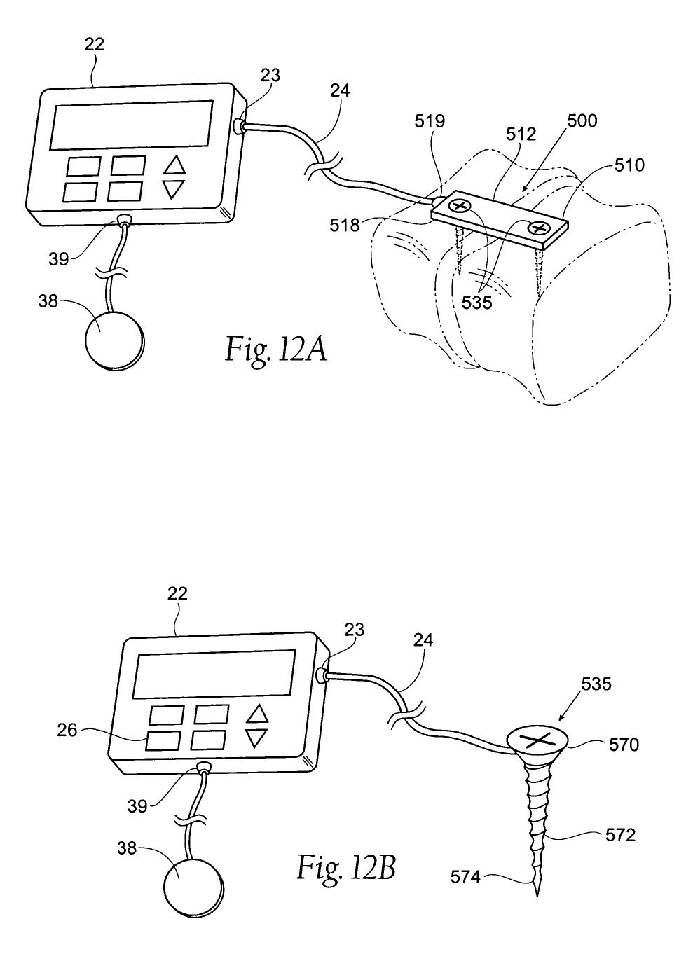

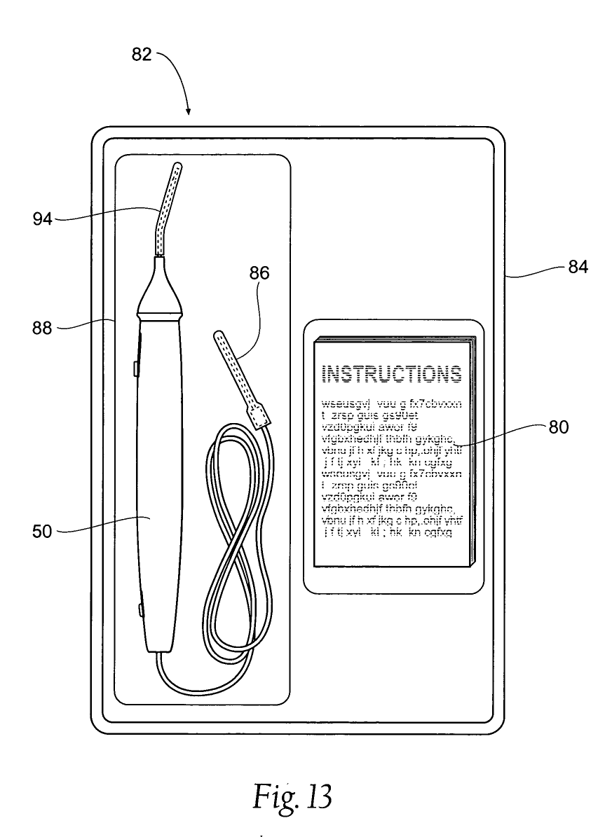

This embodiment incorporates all the features disclosed in the description of the stimulation probe 50, 100, except this embodiment comprises the additional feature of providing an “energized” fixation device or tool. FIG. 12A shows a fixation device 500 removably coupled to a stimulation control device 22. In the embodiment shown, the fixation device 500 includes a rectangularly shaped body 512 that also serves as an operative element, such as electrode 510. The fixation device 500 may take on an unlimited number of shapes as necessary for the particular procedure taking place. Pedicle screws 535 may be used to secure the fixation device to the bony structure. The electrode 510 performs the fixation feature in electrical conductive contact with a hard structure (e.g., bone).

The fixation device 500 or wrench or screwdriver for placing the fixation device desirably includes a plug-in receptacle 519. The fixation device 500 may take on an unlimited variety of shapes and sizes depending on the specific surgical procedure being performed. In this configuration, the lead 24 electrically connects the electrode 510 to the stimulation control device 22 through plug-in receptacle 519.

In one embodiment, the fixation device 500 is mono-polar and is equipped with the single electrode 510. In the mono-polar mode, the stimulation control device 22 includes a return electrode 38 which functions as a return path for the stimulation signal. Electrode 38 may be any of a variety of electrode types (e.g., paddle, needle, wire, or surface), depending on the surgical procedure being performed. The return electrode 38 may be attached to, the stimulation device 22 by way of a connector or plug-in receptacle 39. In an alternative embodiment, the fixation device 500 may be bipolar, which precludes the use of the return electrode 38.

In yet an additional alternative embodiment (see FIG. 12B), the fixation device may be a pedicle screw 535. The pedicle screw 535 is removably coupled to a stimulation control device 22. In the embodiment shown, the pedicle screw 535 includes a head 570 and a shaft 572, which both serve as an operative element, such as electrode 574. The electrode 574 performs the fixation feature in electrical conductive contact with a hard structure (e.g., bone), as the pedicle screw 535 is being positioned within a bony structure. The lead 24 electrically connects the electrode 574 to the stimulation control device 22, through a break-away connection or other similar electrical connective means. The fixation device 535 may take on an unlimited variety of shapes and sizes depending on the specific surgical procedure being performed.

In the mono-polar mode, the stimulation control device 22 includes a return electrode 38 which functions as a return path for the stimulation signal. Electrode 38 may be any of a variety of electrode types (e.g., paddle, needle, wire, or surface), depending on the surgical procedure being performed. In an alternative embodiment, the fixation device 500 may be bipolar, which precludes the use of the return electrode 38.

The present invention includes a method of identifying/locating tissue, e.g., a nerve or muscle, in a patient that comprises the steps of providing a fixation device 500 as set forth above, engaging a patient with the first electrode 510 and the second electrode 38, turning power on to the stimulation control device 22 through the I/O controls 26, causing a stimulation signal 29 to be generated by the stimulation control device 22 and transmitted to the first electrode 510, through the patient's body to the second electrode 38, and back to the stimulation control device 22. The method may also include the step of observing the indicator 126 to confirm the fixation device 500 is generating a stimulation signal. The method may also include the step of observing a tissue region to observe tissue movement or a lack thereof.

### IV. Technical Features

The stimulation control device 22, either alone or when incorporated into a stimulation probe or surgical device, can incorporate various technical features to enhance its universality.

A. Small Size

According to one desirable technical feature, the stimulation control device 22 can be sized small enough to be held and used by one hand during surgical procedures, or to be installed within a stimulation probe or surgical device. The angle of the stimulating tip facilitates access to deep as well as superficial structures without the need for a large incision. Visual and/or audible indication incorporated in the housing provides reliable feedback or status to the surgeon as to the request and delivery of stimulus current.

According to an alternative desirable technical feature, the stimulation control device 22 may also be sized small enough to be easily removably fastened to a surgeon's arm or wrist during the surgical procedure, or positioned in close proximity to the surgical location (as shown in FIG. 7), to provide sufficient audio and/or visual feedback to the surgeon.

B. Power Source

According to one desirable technical feature, power is provided by one or more primary batteries 34 for single use positioned inside the housing and coupled to the control device 22. A representative battery 34 may include a size “N” alkaline battery. In one embodiment, two size “N” alkaline batteries in series are included to provide a 3 volt power source. This configuration is sized and configured to provide an operating life of at least seven hours of operation—either continuous or intermittent stimulation.

C. The Microprocessor/Microcontroller

According to one desirable technical feature, the stimulation control device 22 desirably uses a standard, commercially available micro-power, flash programmable microcontroller 36. The microcontroller 36 reads the controls operated by the surgeon, controls the timing of the stimulus pulses, and controls the feedback to the user about the status of the instrument (e.g., an LED with 1, 2, or more colors that can be on, off, or flashing).

The microcontroller operates at a low voltage and low power. The microcontroller send low voltage pulses to the stimulus output stage 46 that converts these low voltage signals into the higher voltage, controlled voltage, or controlled current, stimulus pulses that are applied to the electrode circuit. This stimulus output stage 46 usually involves the use of a series capacitor to prevent the presence of DC current flow in the electrode circuit in normal operation or in the event of an electronic component failure.

### V. Representative Use of a Stimulation Probe

The stimulation probe 50, 100, as described, make possible the application of a stimulation signal at sufficiently high levels for the purposes of locating, stimulating, and evaluating nerve or muscle, or both nerve and muscle integrity in numerous medical procedures, including, but not limited to, evaluating proximity to a targeted tissue region, evaluating proximity to a nerve or to identify nerve tissue, evaluating if a nerve is intact (i.e., following a traumatic injury) to determine if a repair may be needed, evaluating muscle contraction to determine whether or not the muscle is innervated and/or whether the muscle is intact and/or whether the muscle is severed, and evaluating muscle and tendon length and function following a repair or tendon transfer prior to completing a surgical procedure.

Instructions for use 80 are desirably included in a kit 82 along with a stimulation probe 50. The kit 82 can take various forms. In the illustrated embodiment, kit 82 comprises a sterile, wrapped assembly. A representative kit 82 includes an interior tray 84 made, e.g., from die cut cardboard, plastic sheet, or thermo-formed plastic material, which hold the contents. Kit 82 also desirably includes instructions for use 80 for using the contents of the kit to carry out a desired therapeutic and/or diagnostic objectives.

The instructions 80 guide the user through the steps of unpacking the stimulation probe 50, positioning the electrodes, and disposing of the single use disposable stimulator 50. Representative instructions may include, but are not limited to:
Remove the stimulation probe 50 from sterile package 88.
Remove cover 94 (e.g., a silicone cover) from the operative element 110.
Remove protective cover 86 from the return electrode 131.
Position the return electrode 131 in contact with the patient such that:
   1. The return electrode is desirably positioned in an area remote from the area to be stimulated.
   2. The return electrode is desirably not positioned across the body from the side being stimulated.
   3. The return electrode is desirably not in muscle tissue.
Turn the stimulation probe 500N by moving the power switch 155 from OFF to the 0.5 mA setting (or greater). The stimulation probe 50 desirably is turned ON before the operative element 110 makes contact with tissue.
The indicator 126 will be illuminated yellow (for example) continuously if the stimulation probe 50 is ON, but not in contact with tissue.
Contact tissue with the operative element 110.
Adjust the pulse control 160 gradually to increase the level of stimulation. The indicator 126 will flash yellow indicating that stimulation is being delivered.
A flashing red (for example) indicator 126 means that stimulation has been requested, but no stimulation is being delivered because of inadequate connection of the operative element 110 or the return electrode 131 to the patient tissue. Check the return electrode contact and position, and check the operative element 110 contact and position.
Placing the power switch 155 to the off/standby position will stop stimulation and the visual indictor 126 will be illuminated yellow continuously.
Placing the pulse control 160 at the minimum position will stop stimulation and the visual indictor 126 will be illuminated yellow continuously.
A low/depleted battery 34 will cause the stimulation probe 50 to automatically turn OFF and the visual indicator 126 will not be illuminated. No further use of the stimulator 50 will be possible.
At end of use, move the power switch 155 to the off/standby position and move the pulse control 160 to the minimum position.
Cut off and dispose of the return electrode 131 in an appropriate sharps/biohazard container.
Dispose of the stimulation probe 50 per hospital or facility guidelines.

The problems prior to introduction of embodiments of the device disclosed herein were that current tissue stimulators did not provide an adequate range of stimulus intensity, could not reliably reproduce the stimulus, and did not have an intuitive feel and ergonomic form factor.

An intuitive feel and ergonomic form factor allows a surgeon to operate the device with one hand without diverting attention from the surgical field, which is where the surgeon's attention must be focused at substantially all times. A surgical field perimeter may be conventionally defined physically by a surgical drape, for example. A three-dimensional surgical field may be surrounded by such perimeter, and include a targeted tissue region to receive electrical stimulation. The three-dimensional field further extends away from the animal body, substantially perpendicular to a tangent of the body portion surrounded by the surgical drape. While a method of defining a surgical field has been disclosed, it is to be understood that embodiments of methods according to the present invention may be performed in a surgical field including a targeted tissue region, wherein the surgical field is preferably a maximum size of about 5 centimeters wide, by about 5 centimeters deep, by about 30 centimeters long.

Prior devices are large and bulky and frequently require a remote console controlled by a second physician or technician. This results in the inability by the surgeon to coordinate the application of the stimulus and the observation of the area being stimulated and the response. Such remotely operated units do not afford the surgeon adequate control and are unwieldy and costly to operate because of the extra manpower required.

An embodiment of a hand-held tissue stimulation system including features as disclosed herein is advantageous to allow a surgeon to remain focused and concentrated on a targeted tissue region, while at the same time altering electrical stimulation parameters and receiving substantially simultaneous or instantaneous situational feedback, both of which may occur within an established operating, or surgical, field. Procedures in which devices such as that disclosed may be performed under a microscope upon structures of very small size; therefore, the ability to manipulate and precisely control such device with only one hand without having to look away from the microscope may be highly desirable because movement of the tip (stimulation probe) by, for example, less than 1 millimeter may completely change the response.

Preferred methods according to the present invention include the placement of an entire handheld electrical stimulation device into a surgical, operating field, manipulating electrical stimulation parameters, and observing visual feedback from the device. The manipulation of electrical stimulation parameters preferably occurs manually within the surgical, operating field, and the feedback is preferably generated within the operating field, which may consist of visual feedback to be perceived by a surgeon's peripheral vision or audio feedback to be heard by the surgeon.

Several features of the present invention assist in providing an improved, intuitive, successful experience for the surgeon.

One aspect of embodiments of the present invention adding to ergonomic methods of intraoperative neural stimulation are the user operable controls 155, 160. Both the type and positioning of the user operable controls 155, 160 are thought to improve operability. First, the housing 112 and/or overmolding 68 has a circumferential surface 73 formed about a housing longitudinal axis 75, as can be seen in FIG. 5. At least one user operable control, such as the pulse control device 160, preferably protrudes radially away from the housing longitudinal axis 75 beyond the housing surface 73 to enable the pulse control device 160 to be located by touch.

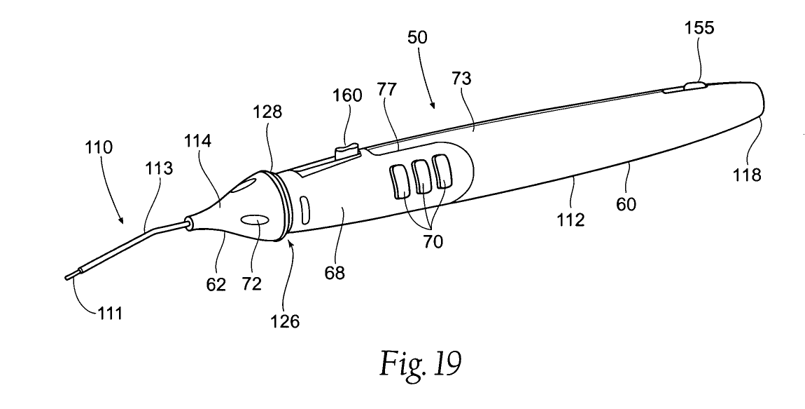

Another aspect of embodiments of the present invention that assists an ergonomic intraoperative neural stimulation is the gripping base portion 60. As noted above, gripping base portion 60 of the housing 112 may also include an overmolded portion 68. The overmolded portion 68, which is preferably formed from a material that is softer than the housing 112, may comprise the full length of the gripping base portion 60, or only a portion of the gripping base 60, such as that shown on FIG. 19. The softer overmolded portion 68 may include one or more features, such as dimples or ribs 70, as shown, to improve the gripping, control, and stability of the stimulation probe 50 within the surgeon's hand. The ribs 70 are preferably disposed distally from at least one user operable control, such as the pulse duration control 160. Additionally, the overmolded portion 68 may include a location channel 77, which may extend longitudinally towards the user operable control 160, and be circumferentially aligned with the user operable control 160. Accordingly, while the overmolded portion 68 is generally provided to improve grip, the location channel 77 may serve to provide the surgeon with the ability to, by touch only, determine in which rotational position the device is in his or her hand. Furthermore, because the softer, overmolded portion 68 is not present in the channel 77, and the preferably harder plastic housing 112 is located therein, a surgeon's finger may more readily slide along the channel 77 towards the user operable control 160. The result is that the surgeon is provided with a mechanism to more easily determine both the rotational position and the location of the user operable control 160 to be adjusted during stimulation. It is preferred that, if the location channel 77 is provided, that the ribs 70 are provided on either side of the channel 77 to allow engagement with, for example, a surgeon's thumb on one side and another finger, such as the middle or ring finger, on the other side. The overmolded portion 68 may comprise the same or similar material as the thermoplastic elastomer material used for the outer portion 66 of the flexible nose cone 62.

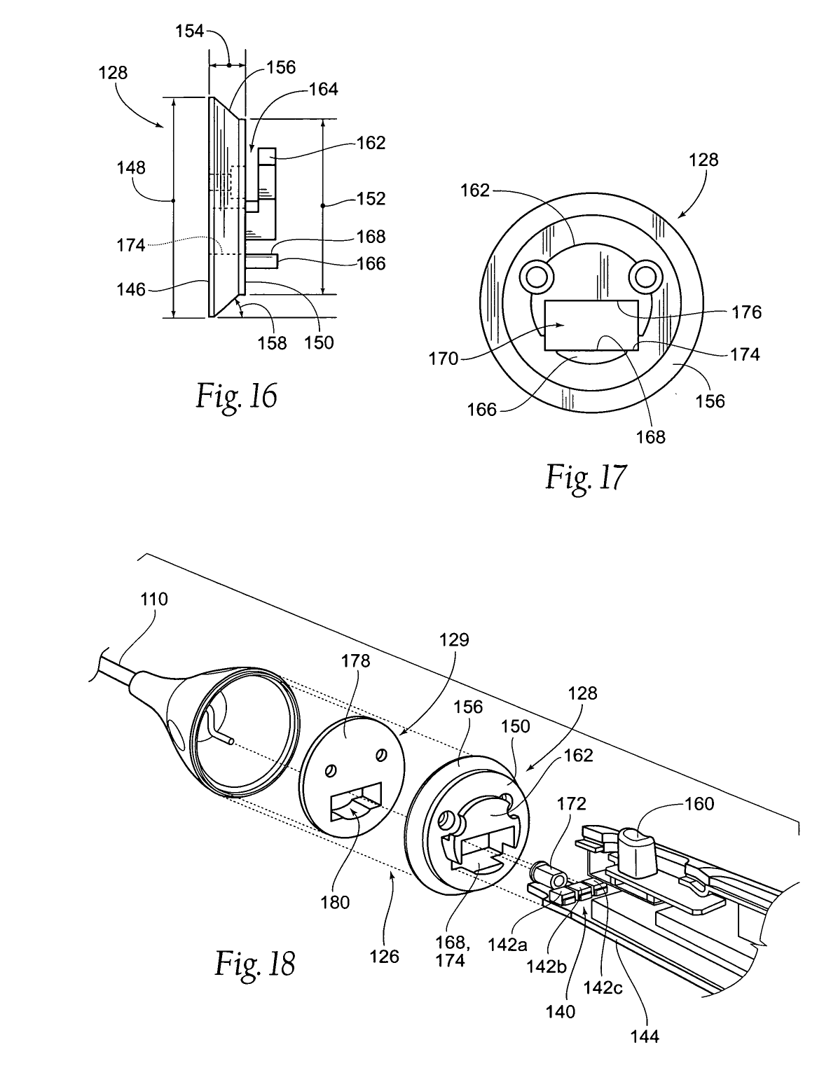

Referring now to FIGS. 16-18, yet another aspect of embodiments of the present invention, which assist in improved intraoperative stimulation, is the visual indicator 126. Preferably, the visual indicator 126 is located between at least one user operable control, such as the pulse duration control 160, and the stimulating tip 111 and/or the operative element 110. Even more preferably, the visual indicator 126 is located between all user operable controls and the stimulating tip 111. Such positioning places the visual indicator 126 in a surgeon's peripheral field of vision during an intraoperative stimulation, allowing the surgeon to remain focused on the targeted tissue region.

A preferred visual indicator 126 includes a light ring 128 and at least one light source 140, which may comprise a light emitting diode (LED) 142. More preferably, the visual indicator 126 includes a plurality of light sources 140, which may include a plurality of LED's 142a, 142b, 142c arranged in a desirable pattern, preferably surface mounted to a printed circuit board 144. The light ring 128 has a proximal surface 146, which is preferably a planar surface having a first diameter 148. The light ring 128 has a distal surface 150, opposed from the proximal surface 146. The distal surface 150 is preferably a planar surface having a second diameter 152. The second diameter 152 is preferably smaller than the first diameter 148. The distal surface 150 is preferably at least substantially parallel to the proximal surface 146, and disposed at a ring thickness 154 therefrom. The light ring 128 further preferably includes an annular surface 156 disposed between the proximal surface 146 and the distal surface 150, where the annular surface 156 is oriented at an angle 158 that is neither parallel to the housing axis 75, when the device is assembled, nor orthogonal thereto.

The annular surface 156 may extend completely between the proximal surface 146 and the distal surface 150, or only partially therebetween, as shown in FIG. 16. The annular surface 156, disposed at such oblique angle, assists in transmitting a majority of the light generated by the light source 140 distally, away from the stimulating tip 111.

The light ring 128 further includes support structure, to assist in coupling the ring 128 to the housing 112. Preferably, the ring 128 is provided with a retention flange 162 spaced from but preferably parallel to the distal surface 150. The retention flange 162 is coupled to the distal surface 150 and provides a mounting channel 164 between the flange 162 and the distal surface 150. The mounting channel 164 is adapted to receive a portion of the housing 112 when the device is assembled so as to prevent longitudinal and radial displacement, and also rotation, of the ring 128 with respect to the housing 112. Provided in addition to the retention flange 162 is an optional circuit board support tab 166, extending preferably substantially orthogonally from the distal surface 150, providing an extended circuit board support surface 168.

A plurality of apertures may be provided through the light ring 128. A first aperture 170 may provide a passageway to allow electrical coupling of the operative element 110 to the electrical stimulation generation circuitry 22 contained within the housing 112, such as through a friction-fit connector 172 mounted on the printed circuit board 144. The same opening 170 preferably provides a light shroud including a circuit board support surface 174 and a light receiving surface 176. Once assembled onto the device, the light source(s) 140 are disposed substantially within the opening 170 between the circuit board support surface 174 and the light receiving surface 176. The light receiving surface 176 is preferably disposed substantially perpendicular to the dominant viewing angle of the light source(s) 142. For instance, a preferred light source 142 is a surface mount LED, such as PLCC surface mount LEDs available from Avago Technologies. Such LEDs have a dominant viewing angle which is substantially perpendicular to the surface onto which the LEDs are mounted. The LEDs have a viewing angle of about 120 degrees. A preferred arrangement of light sources 142 includes the use of three light sources 142a, 142b, 142c. The three light sources are preferably aligned in a row on the printed circuit board 144. While one or more colors of LEDs could be used, it is preferred that at least two colors are provided. For instance, light sources 142a and 142c could be amber LED's having a dominant wavelength of about 592 nanometers, and light source 142b could be a red LED having a dominant wavelength of about 630 nanometers. While the LEDs could be arranged in any desirable order, it is preferred to provide a balanced light output. Accordingly, it may be preferred to dispose the two amber LEDs 142a, 142c, one on either side of the red LED 142b, thereby preventing any light bias and providing a balanced light output. Thus, the LEDs 142a, 142b, 142c are disposed on the printed circuit board 144, which is inserted into the aperture 170 to rest adjacent the circuit board support surface 174.

Directionality of light provided through the light ring 128 is thought to improve the usefulness of devices according to the present invention, especially in situations where light indications are to be observed in a surgeon's peripheral field of vision. While part of the light directionality is provided by the design of the light ring 128, itself, such directionality may be enhanced by the inclusion of a reflective element 129 disposed adjacent to or formed as a part of the light ring 128. The reflective element 129 preferably includes a white distal surface 178, which is a preferably substantially planar surface adapted to rest adjacent the proximal surface 146 of the light ring 128. The distal surface 178 of the reflective element 129 aids in directing light through the light ring 128 and out of the annular surface 156 thereof. The reflective element 129 also preferably includes an aperture 180 therethrough, which may mate with the aperture 170 formed through the light ring 128 to allow connectivity of the operative element 110 to the stimulation generation circuitry 22.

The light ring 128 is preferably formed as a unitary member including the proximal surface 146, the distal surface 150, and the optional retention flange 162 and circuit board support tab 166. The light ring 128 is preferably formed from a substantially optically clear material and preferably has a highly polished annular surface 156, so as to provide a desired transmittance.

The operation of the visual indicator 126 is informational to a user. The visual indicator 126, through various color and flash pattern indications, informs the surgeon more than just when a nerve has been stimulated, it informs the surgeon whether the electrical flow path of the stimulation signal is complete. That is, when the flow path is complete, the visual indicator 126 informs the surgeon that the stimulation circuitry 22 is generating a stimulation signal, the stimulation signal is being delivered to the stimulating tip 111, the stimulation signal is passing through tissue at the targeted tissue region, and that the stimulation signal is being received at the stimulation circuitry 22 through the return electrode 130. This status of the device, in combination with observation of the neural response to the applied stimulation, provides the surgeon the ability to assess the health of a nerve.

The visual indicator 126 provides stimulation system status conditions, the status conditions including (i) confirmation of electrical power provided to the stimulation generation circuitry 22, such as by continuous amber or yellow lighting of LEDs 142a and 142c, (ii) confirmation of stimulation availability at the stimulating tip 111, such as by flashing of either the amber LEDs 142a, 142c or the red LED 142b, and (iii) confirmation of stimulation delivery to the targeted tissue region, such as by flashing of either the amber LEDs 142a, 142c or the red LED 142b. The power supply, including the battery 34, is configured such that, once power is supplied to the circuitry 22, the visual indicator 126 will illuminate for confirmation of such power provision, and the visual indicator 126 preferably cannot be turned off, until the battery charge dissipates completely, or is otherwise removed from the device.

Still another aspect of embodiments according to the present invention adding to the usability of intraoperative stimulation is the provision of stimulation parameters to allow for continuous stimulation during translation or relocation of the stimulating tip 111, while maintaining contact with tissue or even during stimulation parameter adjustment without causing tissue damage. Indeed, stimulation pulse trains may continually be generated, at a desired frequency, while the electrode is in contact with a targeted tissue region, as well as during periods of stimulation parameter adjustment. Thus, a method according to the present invention includes providing an electrical stimulation device, such as embodiments described herein, including an operative element having an electrode stimulating tip disposed thereon. The electrical stimulation is started at a desired, predetermined, or established frequency, where electrical stimulation pulses having specified duration are supplied to the electrode at such frequency. Electrical stimulation is continued at the desired frequency while one or more electrical stimulation parameters, such as amplitude and duration, are adjusted. Alternatively, or additionally, electrical stimulation is applied to a targeted animal tissue region during the adjustment or alteration of the electrical stimulation parameters. Alternatively, or additionally, electrical stimulation is applied to a targeted animal tissue region during translation of the electrode across and in electrically communicative contact with the tissue region. Thus, while electrical stimulation is being delivered at a frequency, either or both of the following actions may be performed: (i) translating or moving of the electrode in contact with the tissue region; and (ii) adjusting or altering electrical stimulation parameters, such as amplitude and/or pulse duration. All of these actions may be performed while receiving feedback from the indicator 126 provided on the device. Accordingly, while allowing a surgeon to maintain focus on a targeted tissue region, methods according to the present invention improve the ergonomic and intuitive use of an electrical stimulator, by allowing substantially simultaneous electrode/tissue translation, adjustment of electrical stimulation parameter(s) and informational feedback.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

We claim:

1. A method of applying electrical stimulation to animal tissue, the method comprising the steps of:
identifying a three-dimensional surgical field comprising a targeted tissue region of an animal;
providing a device comprising:
a housing extending along a housing longitudinal axis between a housing proximal end and a housing distal end;
electrical stimulation generation circuitry at least substantially contained within the housing;
one or more user operable controls coupled to the housing and in operational communication with the electrical stimulation generation circuitry;
an operative element extending from the housing proximal end, the operative element comprising an electrode operatively coupled to the electrical stimulation generation circuitry; and
a power supply disposed at least substantially within the housing, the power supply being electrically coupled to the electrical stimulation generation circuitry;
introducing the entire housing into the surgical field; and
applying electrical stimulation from the electrode to a targeted tissue region located within the surgical field.

2. A method according to claim 1, wherein the surgical field extends from the targeted tissue region for a maximum length of thirty centimeters.

3. A method according to claim 1, wherein the surgical field is about 20 to about 50 millimeters wide, about 18 to about 22 centimeters long, and about 20 to about 50 millimeters deep.

4. A method according to claim 1, wherein the surgical field is at least substantially sterile.

5. A method according to claim 4, wherein the device is at least substantially sterile.

6. A method according to claim 1, wherein the stimulation circuitry is contained entirely within the housing.

7. A method according to claim 1, wherein the power supply is contained entirely within the housing.

8. A method according to claim 1, further comprising the step of carrying the device by a single human hand.

9. A method according to claim 8, further including a step of manipulating, with the single human hand, the device to change an electrical stimulation parameter.

10. A method according to claim 9, wherein the manipulating step is carried out while the entire housing is positioned within the surgical field.

11. A method according to claim 9, wherein the manipulating step occurs during the step of applying electrical stimulation from the electrode to the targeted tissue region located within the surgical field.

12. A method according to claim 1, the device further comprising an electronic visual indicator operatively coupled to the stimulation circuitry.

13. A method according to claim 12, further comprising a step of observing a first visual indication provided by the visual indicator, the first visual indication being indicative of electrical power supplied to the stimulation circuitry by the power supply.

14. A method according to claim 13, further comprising a step of observing a second visual indication provided by the visual indicator, the second visual indication being indicative of electrical stimulation flowing at least partially through the targeted tissue region.

15. A method according to claim 14, wherein the first visual indication is an illumination of a first color and the second visual indication is an illumination of a second color, the second color being different from the first color.

16. A method according to claim 12, wherein the visual indicator is an illumination device that is radially visible from 360 degrees around the longitudinal axis of the handle.

17. A method according to claim 16, wherein the visual indicator is situated between one of the user operable controls and the electrode.

18. A method according to claim 1, wherein the applying step is carried out while the entire housing is positioned within the surgical field.

19. A method according to claim 1, wherein the targeted tissue region comprises at least one of muscle tissue and nerve fibers.

20. A method of applying electrical stimulation to a targeted animal tissue region, the method comprising the steps of:
receiving, into a single human hand, a device comprising:
a housing extending along a housing longitudinal axis between a housing proximal end and a housing distal end;
electrical stimulation generation circuitry at least substantially contained within the housing;
one or more user operable controls coupled to the housing and in operational communication with the electrical stimulation generation circuitry;
an operative element extending from the housing proximal end, the operative element comprising an electrode operatively coupled to the electrical stimulation generation circuitry; and
a power supply disposed at least substantially within the housing, the power supply being electrically coupled to the electrical stimulation generation circuitry;
generating a first indication, which is indicative of electrical power being supplied to the electrical stimulation generation circuitry;
providing a first electrical stimulation from the stimulation generation circuitry to the electrode;
generating a second indication, which is indicative of the first stimulation is being provided to the stimulating tip and is further indicative of the first stimulation being prevented from being received by the device through a return electrode;
applying the first electrical stimulation to a targeted animal tissue region; and,
generating a third indication, which is indicative of the first stimulation being received by the device through the return electrode.

21. A method according to claim 20, wherein all of the indications are generated within a three-dimensional surgical field including the targeted tissue region.

22. A method according to claim 21, wherein all indications are visual indications.

23. A method according to claim 22, wherein the visual indications are generated by a visual indicator disposed between the stimulating tip and all user operable controls.

24. A method according to claim 23, wherein the visual indicator comprises a light ring having a proximal surface and a distal surface coupled by an oblique annular surface.

25. A method according to claim 24, wherein the light ring proximal surface has a substantially circular perimeter.

26. A method according to claim 25, wherein the light ring distal surface has a substantially circular perimeter.

27. A method according to claim 26, wherein the distal surface perimeter is smaller than the proximal surface perimeter.

28. A method according to claim 27, wherein the generating steps include the step of transmitting visible light through the light ring annular surface and distally from the electrode.

29. A method according to claim 24, wherein the visual indicator further comprises a reflective element mounted adjacent the proximal surface of the light ring.

30. A method of manipulating a handheld electrical stimulator within a surgical field, the method comprising the steps of:
receiving, into a single human hand, a device comprising:
a housing extending along a housing longitudinal axis between a housing proximal end and a housing distal end;
electrical stimulation generation circuitry at least substantially contained within the housing;
one or more user operable controls coupled to the housing and in operational communication with the electrical stimulation generation circuitry;
an operative element extending from the housing proximal end, the operative element comprising an electrode operatively coupled to the electrical stimulation generation circuitry; and
a power supply disposed at least substantially within the housing, the power supply being electrically coupled to the electrical stimulation generation circuitry,
wherein the housing includes a gripping portion comprising a location channel extending longitudinally distally from one of the user operable controls;
using an index finger of the hand, identifying the location channel;
locating one of the user operable controls proximal the location channel; and
manipulating the located user operable control.

31. A method according to claim 30, wherein the locating step is performed by sliding the index finger proximally along the location channel.

32. A method according to claim 30, wherein all steps except the receiving step are performed within a three-dimensional surgical field.

33. A method according to claim 30, wherein the operative element comprises an angled portion, thereby defining an operative element plane, and the location channel and located user operable control are disposed substantially coplanar with the operative element plane.
